(19) United States
(12) Patent Application Publication
(10) Pub. No.: US 2006/0225734 A1
(43) Pub. Date: Oct. 12, 2006

(54) Filter for oxygen delivery systems

(75) Inventors: John E. Sagaser, Boise, ID (US); Steve Hatten, Boise, ID (US)

Correspondence Address: DYKAS, SHAVER & NIPPER, LLP, P.O. BOX 877, BOISE, ID, 83701-0877, US

(73) Assignee: Ox-Gen Inc., Boise, ID (US)

(21) Appl. No.: 11/402,219

(22) Filed: Apr. 10, 2006

Related U.S. Application Data
(60) Provisional application No. 60/669,638, filed on Apr. 8, 2005.

(51) Int. Cl. G08B 3/00; A62B 9/00; A61M 16/00; G08B 5/00

(52) U.S. Cl. 128/202.22; 128/205.29; 128/201.25

(57) ABSTRACT

The present invention is a filter for use with an oxygen delivery system. The filter includes a made up of a housing, with a pair of inlets and which defines a variety of internal features and structures which will be described here below. The filter operates by conducting the oxygen from a source through the first inlet and into a receiving chamber form this receiving chamber through a foam and charcoal filter and back out through an outlet for use by a user. This filter and system provides a way for delivering cleaned, filtered oxygen to the intended user through a delivery device such as a mask or a nasal canula.

### CROSS-REFERENCE TO RELATED APPLICATIONS

This application claims the priority date of a previously filed provisional application entitled SPORT-OX SKI 2 FILTER, filed by these same inventors Apr. 8, 2005, with application Ser. No. 60/669,638. The contents of this application are herein incorporated by reference.

### FIELD OF THE INVENTION

The present invention generally relates to apparatuses for delivering oxygen from a source to a user and more particularly to a filter for such oxygen delivery systems.

### BACKGROUND OF THE INVENTION

There are a number of situations in which a source of oxygen would be an essential lifesaving tool. This could include a situation where a person is in a burning building and a supply of oxygen, even if only for a few minutes, would increase his or her chances of escape from the smoke filled building. This could apply to office workers, rescue personnel or police.

Another situation in which emergency oxygen would be useful is in response to an emergency situation, such as an environment filled with poisonous gases. This could occur in a chemical plant from a rupture of a tank, or could occur on a battlefield from the use of chemical weapons. In such a case, having a quickly available supply of oxygen that has been conveniently stored and has a long shelf life would be a lifesaver. Other situations in which an emergency supply of oxygen would be useful, would include use by pilots who may need to clear their head when flying at a higher elevation, first-aid situations in which oxygen may need to be administered in the field before the person is picked up by oxygen equipped rescue personnel, at home where a person may wish to administer oxygen in response to shortness of breath, heart arrhythmia, heart attack or stroke.

The prior art includes many oxygen generation devices. Many of them involve a rigid canister in which oxygen gas is compressed, and from which it can be released for breathing. Other prior art oxygen generation systems are reaction vessels in which chemicals of various types can be added in order to set up a reaction that generates oxygen. The problem with compressed oxygen is that these systems are expensive, heavy and not practical for most people to have on hand or for field situations. Devices based on a reaction vessel are impractical if the reaction vessel is bulky and hard to carry, and if the chemicals take any more than the absolute minimum of time and effort to add and mix for use. A person cannot hold their breath very long while preparing such a canister, measuring ingredients, and adding the ingredients. A reaction vessel which takes more than ten (10) seconds to access, activate, and begin receiving oxygen is not very effective. One that takes several minutes to access, activate and begin receiving oxygen is not particularly practical in the situations that are described above.

A portable emergency oxygen generation system needs to be small in size, have a long shelf life, be easy to activate, but which does not activate accidentally, and must generate breathable oxygen within a few seconds of activation. Anything that takes more than even five seconds is not effective in certain situations. It must also generate a sufficient volume of oxygen for a sufficient amount of time to be useful.

While oxygen can be delivered from a variety of sources, the generation or storage of oxygen in many instances produces oxygen that has contaminants such as water, chemicals and other items that are suspended within the oxygen. Inhaling these items can cause not only irritation to the mucosal linings of the inhaler, but can also cause damage to the lungs and upper respiratory tract of the inhaler. Therefore, what is needed is a filter for such oxygen delivery systems.

### SUMMARY OF THE INVENTION

The present invention is a filter for use with an oxygen delivery system. The filter is made up of a housing that defines a pair of inlets and a variety of internal features and structures, which will be described here below. The inlets of the housing are positioned on the housing and provide a passageway whereby a first inlet is connected to a receiving chamber defined within the housing, and a second inlet is connected to a filter chamber exit passageway, which is also defined within the housing. The receiving chamber and the filter chamber are interconnected by an air chamber passageway. The filter chamber has a first foam pad, a preselected quantity of activated charcoal, an air diverter plate, and a second foam pad positioned within the filter chamber whereby oxygen passing through the chamber must come into contact with and be affected by all of these materials.

The filter operates by conducting the oxygen from a source through the first inlet and into the receiving chamber. At this position, the flow of air is slowed by the physical limitations of the chamber and liquids such as water, which may be suspended in the airflow, are precipitated and fall out of the oxygen. This lighter oxygen then rises and pushes the dewatered oxygen out of the receiving chamber, through the air chamber passageway and into the filter chamber. In the filter chamber the dewatered oxygen is passed through the first foam pad, which acts to prevent liquids from entering the remaining portions of the filter. The oxygen is then passed through the charcoal to remove other contaminates by absorption as well as any remaining moisture, fine particulates or other matter that is suspended within the oxygen. After passing through the charcoal, the flow of oxygen is reduced as the air diverter plate causes the oxygen to re-circulate, and percolate among the charcoal. The only escape of air out of the filter chamber occurs through the aperture in the diverter plate. After passing through the aperture in the diverter plate, oxygen is then passed through the second foam pad at the opposite end of the filter chamber and exits the filter through the filter chamber exit passageway and the second inlet, which are formed parts of the housing. From this second inlet, a conduit can be connected so as to deliver the cleaned, filtered oxygen to the intended user through a delivery device such as a mask or a nasal canula.

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other features and advantages of the present invention will become readily apparent to those skilled in this art from the following detailed description describing preferred embodiments of the invention, simply by way of illustration of the best mode contemplated by carrying out my invention. As will be realized, the invention is capable of modification in various obvious respects all without departing from the invention. Accordingly, the drawings and description of the preferred embodiments are to be regarded as illustrative in nature, and not as restrictive in nature.

### BRIEF DESCRIPTION OF THE DRAWINGS

FIG. 1 shows a cutaway view of a preferred first embodiment of the present invention.

FIG. 2 shows a front plant view of the air deflector plate of the present invention.

FIG. 3 shows an end view of the second end of the invention.

FIG. 4 shows an end view of the first end of the invention.

FIG. 5 shows a perspective view of the present invention described above.

FIG. 6 shows a perspective view of the filter of the present invention in use upon a first type of an oxygen generation and delivery system.

FIG. 7 shows a perspective view of the filter of the present invention in use upon a second type of an oxygen generation and delivery system.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

The present invention is a filter for oxygen delivery systems and systems that employ and utilize the filter such as the one that is described herein. In the following description and in the figures, like elements are identified with like reference numerals. The use of “or” indicates a non-exclusive alternative without limitation unless otherwise noted. The use of “including” means “including, but not limited to,” unless otherwise noted.

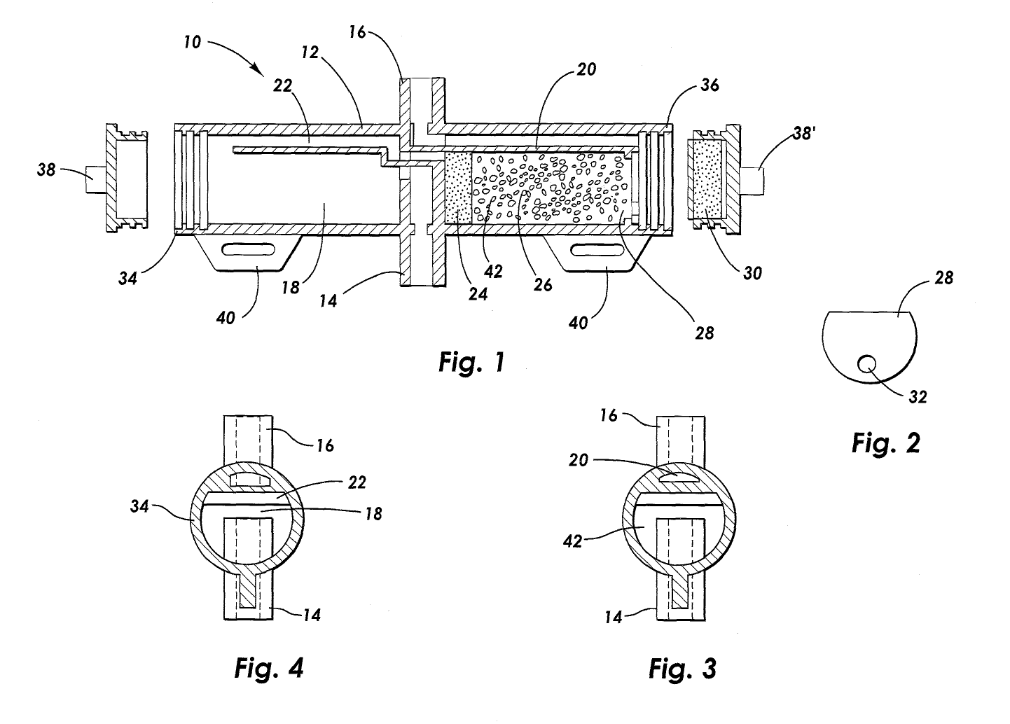

A variety of views of the present invention are set forth in the attached drawings FIGS. 1-7 and in the following description of these drawings. Referring first to FIG. 1, a cutaway view of the preferred embodiment of the invention is shown. FIG. 1 shows a filter 10 for use with an oxygen delivery system. The filter 10 is made up of a housing 12, which in the demonstrated preferred embodiment of the invention, is a generally tubular shaped outer body 12 with a first end 34 and extending along a length 12 to a second end 36. The first and second ends 34, 36, are adapted to receive sealable end caps 38, 38′ within the first and second ends 34, 36, so as to seal the first and second ends 34, 36. The generally tubular shaped outer body 12 further defines a pair of generally symmetrical inlets 14, 16, which are in the preferred embodiment of the invention, positioned on generally opposite sides of the generally tubular shaped body 12, at a location generally equidistant between the first and second ends 34, 36.

These inlets include a first inlet 14 that provides an entrance to a receiving chamber 18 that is defined within the housing 12 and a second inlet 16 that is connected to and provides access to a filter chamber exit passageway 20. The receiving chamber 18 and the filter chamber 42 are interconnected by an air chamber passageway 22. Within the filter chamber 42 a variety of filtration items are placed. These include a first foam pad 24, activated charcoal 26, an air diverter plate 28, and a second foam pad 30. In this demonstrated embodiment, the second foam pad 30 is shown as being located within the sealable end cap 38′, however, it is to be distinctly understood that the invention is in no way limited to this embodiment. The air diverter plate 28, which separates the charcoal 26 from the second foam pad 30, defines an aperture 32 that provides the only outlet from the filter chamber 42.

In use, oxygen from a source enters the housing 12 through the first inlet 14 and passes into the receiving chamber 18, wherein liquids such as water that is suspended or mixed with the oxygen initially are removed. The so-called dewatered oxygen then rises within the receiving chamber 18 to the air chamber passageway 22. The air chamber passageway 22 conducts the oxygen into the filter chamber 42, through the first foam pad 24, which also acts to prevent liquids from entering the remaining portions of the filter chamber 42. After passing through this first foam pad 24, the oxygen then passes into the charcoal 26 where it percolates. This percolation of oxygen through this media, removes contaminates by absorption including any remaining moisture, fine particulates or other matter that is suspended within the oxygen. Passage out of the charcoal portion of the filter chamber 42, is limited to that quantity of oxygen that is enabled to pass through the aperture 32 in the air diverter plate 28. After passing through this aperture 32, the oxygen is routed through a second foam pad 30 that cleanses any particulate charcoal from the oxygen and the oxygen is then transported into the filter chamber exit passageway 20. From this filter chamber exit passageway 20, the oxygen can be passed out of the filter through the second inlet 16. Preferably, this second inlet is connected to a conduit that functions to transport this oxygen to a user. In the preferred embodiment of the invention, the outer generally tubular body 12 also includes an adapter portion 40 that is adapted to connect with a portion of an oxygen generation source.

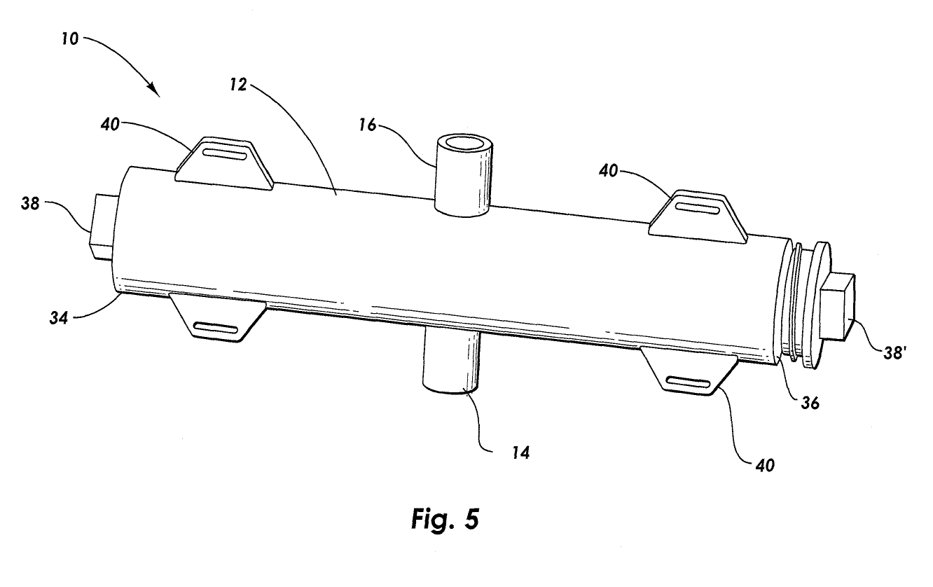

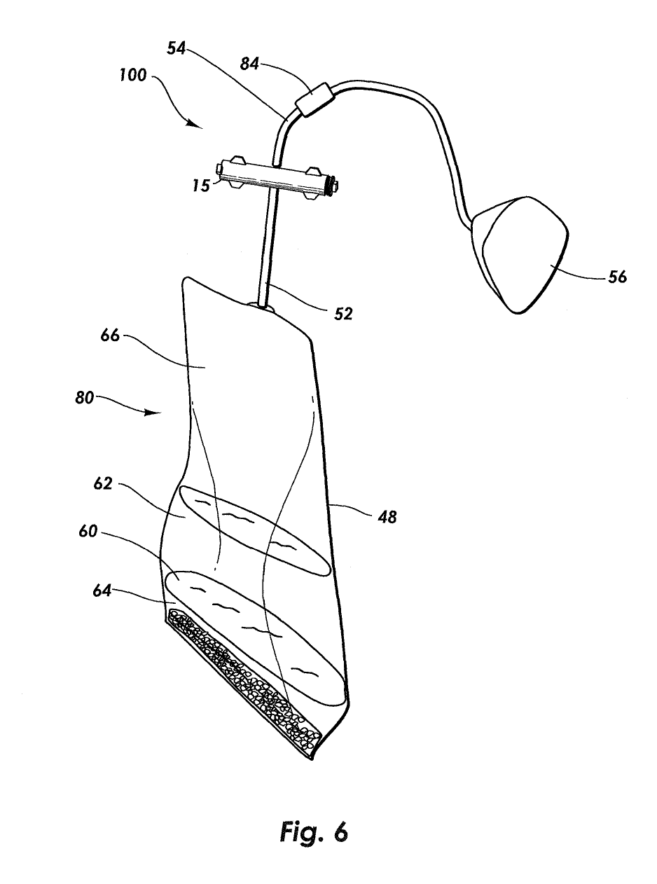

FIGS. 2-5 show various views of the features described above. FIG. 2 shows a front plan view of the air deflector plate 28 that is described above. FIGS. 3 and 4 show end views of housing 12 looking from the second and first ends of the device respectively. FIG. 5 shows a perspective view of the present invention described here above.

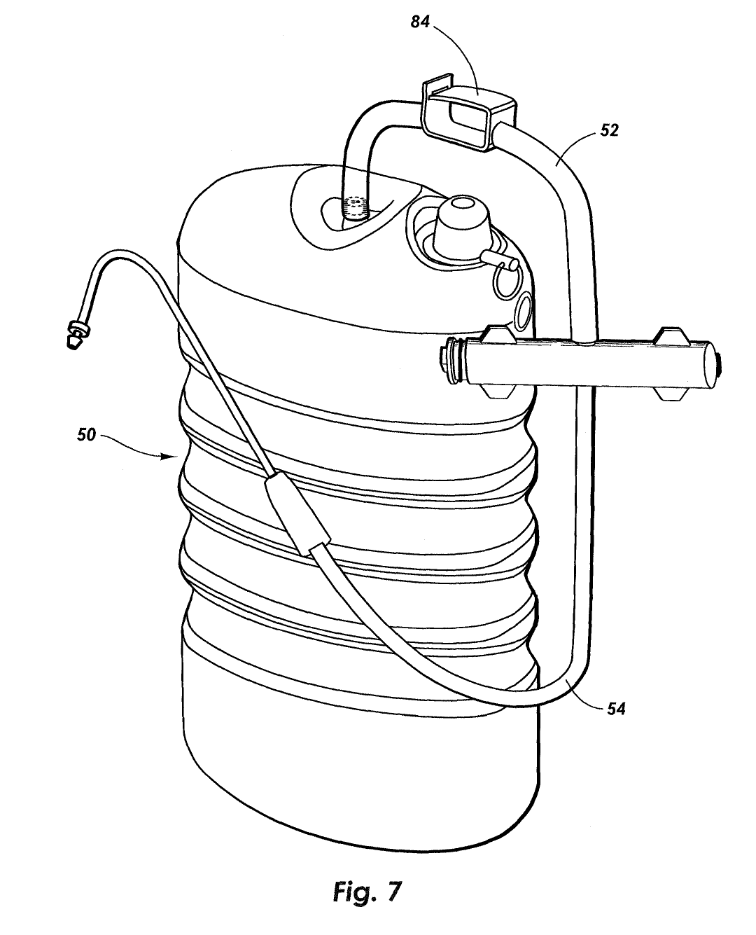

The filter of the present invention can be utilized on a variety of types of oxygen delivery systems including portable systems, wherein oxygen is produced by chemical reaction in a closed container. A description of the use of such a filter 10 in conjunction with various types of these systems is set forth hereafter, and is shown in FIGS. 6 and 7.

FIGS. 6 and 7 show the use of the present invention in conjunction with a compressible type of oxygen generation system as a source for oxygen. FIG. 6 shows an oxygen delivery system made up of a source of oxygen 50, a first conduit 52 adapted to deliver oxygen from the source to the filter 10, a filter 10 having the features that were described above and a second conduit 54 that is configured to deliver oxygen from the filter 10 to a user. In this preferred embodiment of the invention, the second conduit 54 includes a clamp 84 for regulating the flow of air out of the filter 10 and into the delivery mask 56. While a mask 56 is shown in this present preferred embodiment of the invention, it is to be distinctly understood that the invention is not limited thereto, but may also be variously embodied to include a variety of other additional features and delivery devices as well.

The present invention is compatible and may be utilized with a variety of types of oxygen producing gas sources. In the example shown in FIGS. 6 and 7, the oxygen source 50 is a flexible bladder 58 constructed of a resilient material configured to hold a quantity of an inner wall material 60. The inner wall material 60 configured to define at least two chambers, a first chamber 62 configured to hold a selected quantity of a first selected material, and a second chamber 64 configured to hold a selected quantity of a second selected material therein. These materials are selected so that oxygen is produced when the first material and the second materials are mixed. The inner wall material 60 configured so as to allow passage of the materials between the chambers 62, 64, when a selected amount of pressure is applied to the inner wall 60. In the preferred embodiment shown in FIG. 6, the inner wall material 60 also defines a third chamber 66; this third chamber 66 being configured to hold a selected oxygen producing material therein.

The present invention provides a way of producing and delivering clean quality oxygen to a user that is safe, convenient and transportable. While there is shown and described the present preferred embodiment of the invention, it is to be distinctly understood that this invention is not limited thereto, but may be variously embodied to practice within the scope of the following claims. From the foregoing description, it will be apparent that various changes may be made without departing from the spirit and scope of the invention as defined by the following claims.

What is claimed is:

1. A filter for use with an oxygen delivery system, said filter comprising:
a housing, said housing having a pair of inlets, and defining a variety of internal features and structures; said pair of inlets comprised of a first inlet connected to a receiving chamber defined within the housing, and a second inlet connected to a filter chamber exit passageway defined within the housing; the receiving chamber and the filter chamber interconnected by an air chamber passageway; the filter chamber having a first foam pad, charcoal, an air diverter plate, and a second foam pad positioned within the filter chamber, with an aperture defined within the air diverter plate;
whereby oxygen from a source enters the first inlet and passes through the receiving chamber and the air chamber passageway, and into the filter chamber, where the oxygen is passed through the first foam pad, which acts to prevent liquids from entering the remaining portions of the filter, the charcoal to remove other contaminates by absorption as well as any remaining moisture, fine particulates or other matter that is suspended within the oxygen, through the aperture in the air diverter plate, through the second foam pad and exits the filter through the filter chamber exit passageway and the second inlet.

2. The filter of claim 1, wherein said housing is a generally tubular shaped outer body having a first end and extending along a length to a second end.

3. The filter of claim 2, wherein the inlets are generally symmetrically positioned on generally opposite sides of the generally tubular shaped body, at a location generally equidistant between the first and second ends.

4. The filter of claim 2, wherein the first and second ends are adapted to receive sealable end caps within the first and second ends so as to seal the first and second ends.

5. The filter of claim 2 further comprising an adapter portion configured to connect the filter to an oxygen delivery source.

6. An oxygen delivery system comprising:
a source of oxygen;
a first conduit adapted to deliver oxygen from the source to a filter,
the filter comprised of a housing, said housing having a pair of inlets, and defining a variety of internal features and structures; said pair of inlets comprised of a first inlet connected to a receiving chamber defined within the housing, and a second inlet connected to a filter chamber exit passageway defined within the housing; the receiving chamber and the filter chamber interconnected by an air chamber passageway; the filter chamber having a first foam pad, charcoal, an air diverter plate, and a second foam pad positioned within the filter chamber, an aperture is defined within the air diverter plate;
whereby oxygen from a source enters the first inlet and passes through the receiving chamber and the air chamber passageway, and into the filter chamber, there the oxygen is passed through the first foam pad, which acts to prevent liquids from entering the remaining portions of the filter, the charcoal to remove other contaminates by absorption as well as any remaining moisture, fine particulates or other matter that is suspended within the oxygen, through the aperture in the air diverter plate, through the second foam pad and exits the filter through the filter chamber exit passageway and the second inlet; and
a second conduit configured to deliver oxygen from the filter to a user.

7. The oxygen delivery system of claim 6 further comprising an oxygen delivery mask attached to the second conduit.

8. The oxygen delivery system of claim 6, wherein the oxygen source is a flexible bladder the flexible bladder constructed of a resilient material configured to hold a quantity of an inner wall material, the inner wall material configured to define at least two chambers, a first chamber configured to hold a selected quantity of a first selected material, and a second chamber configured to hold a selected quantity of a second selected material therein, the materials are selected so that oxygen is produced when the first material and the second materials are mixed, wherein the inner wall material is configured to allow passage of the materials between the chambers when a selected amount of pressure is applied to the inner wall.

9. The oxygen delivery system of claim 8, wherein the inner wall further defines a third chamber; this third chamber configured to contain a selected oxygen producing material therein.

10. The oxygen delivery system of claim 6, wherein the filter housing is a generally tubular shaped outer body having a first end and extending along a length to a second end.

11. The oxygen delivery system of claim 6, wherein the filter contains inlets that are generally symmetrically positioned on generally opposite sides of the generally tubular shaped body, at a location generally equidistant between the first and second ends.

12. The oxygen delivery system of claim 6, wherein the first and second ends are adapted to receive sealable end caps within the first and second ends so as to seal the first and second ends of the housing.

13. The oxygen delivery system of claim 6 further comprising a clamp on the first conduit.

14. The filter of claim 6 further comprising an adapter portion configured to connect the filter to the oxygen delivery source.

15. A filter for use with an oxygen delivery system, said filter comprising:
a generally tubular shaped outer body, the generally tubular shaped outer body having a first end and extending along a length to a second end, the first and second ends adapted to receive sealable end caps within the first and second ends so as to seal the first and second ends, the generally tubular shaped outer body further defining a pair of generally symmetrical inlets positioned on generally opposite sides of the generally tubular shaped body at a location generally equidistant between the first and second ends, the pair of inlets comprised of a first inlet which provides an entrance to a receiving chamber, and a second inlet providing access to a filter chamber exit passageway, the receiving chamber and the filter chamber interconnected by an air chamber passageway; the filter chamber comprising a first foam pad, charcoal, an air diverter plate, and a second foam pad the air diverter plate defining an aperture within said air diverter plate;
whereby oxygen from a source enters the filter through the first inlet and passes into the receiving chamber, wherein liquids that may be suspended or mixed with the oxygen are removed, liquid less oxygen then rises within the receiving chamber to the air chamber passageway, the air chamber passageway conducts the oxygen through the foam pad, which also acts to prevent liquids from entering the remaining portions of the filter, the oxygen then passes through the charcoal to remove other contaminates by absorption as well as any remaining moisture, fine particulates or other matter that is suspended within the oxygen, after passing through the first foam pad and the charcoal, oxygen passes through the aperture, and the second foam pad and exits the filter through the filter chamber exit passageway and the second inlet.
